(19) United States
(12) Patent Application Publication
(10) Pub. No.: US 2007/0191442 A1
(43) Pub. Date: Aug. 16, 2007

(54) Aqueous Pharmaceutical Formulations of ER-beta Selective Ligands

(75) Inventors: MARC S. TESCONI, MONROE, NY (US); MANNCHING SHERRY KU, THIELLS, NY (US)

Correspondence Address: Pepper Hamilton LLP/Wyeth, 500 Grant Street, One Mellon Bank Center, 50th Floor, Pittsburgh, PA, 15219-2502, US

(73) Assignee: WYETH, Madison, NJ (US)

(21) Appl. No.: 11/674,496

(22) Filed: Feb. 13, 2007

Related U.S. Application Data
(60) Provisional application No. 60/773,028, filed on Feb. 14, 2006.

(51) Int. Cl. A61K 31/42; B65D 1/09

(52) U.S. Cl. 514/375; 514/657

(57) ABSTRACT

The present invention relates to aqueous formulations of ERβ selective ligands. In some embodiments, the formulations include an ERβ selective ligand, a solubilizer/complexant component, and a pH adjusting component. The invention further provides preparations of the formulations, and uses thereof.

This application claims benefit of priority of U.S. Provisional Application Ser. No. 60/773,028, filed Feb. 14, 2006, which is hereby incorporated by reference in its entirety.

### FIELD OF THE INVENTION

The present invention relates to aqueous formulations of ERβ selective ligands. In some embodiments, the formulations include an ERβ selective ligand, a solubilizer/complexant component, and a pH adjusting component. In some preferred embodiments, the ERβ selective ligand is 2-(3-fluoro-4-hydroxyphenyl)-7-vinyl-1,3-benzoxazol-5-ol or 3-(3-Fluoro-4-hydroxy-phenyl)-7-hydroxy-naphthalene-1-carbonitrile.

### BACKGROUND OF THE INVENTION

This invention relates to formulations for ERβ selective ligands, which are useful as estrogenic agents.

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Womens Health & Gender Based Medicine 8: 1155-1166 (1999), Monk and Brodaty, Dementia & Geriatric Cognitive Disorders 11: 1-10 (2000), Hum and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles Of Molecular Regulation. p 351-361(2000)]. A class of “coregulatory” proteins can also interact with the ligand-bound receptor and further modulate its transcriptional activity [McKenna, et al., Endocrine Reviews 20: 321-344 (1999)]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERβ [Green, et al., Nature 320: 134-9 (1986)]. The second form of the estrogen receptor was found comparatively recently and is called ERβ [Kuiper, et al., Proceedings of the National Academy of Sciences of the United States of America 93: 5925-5930 (1996)]. Early work on ERβ focused on defining its affinity for a variety of ligands and indeed, some differences with ERβ were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERβ is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed some compounds behave as estrogen receptor agonists in some tissues and estrogen receptor antagonists in others. These compounds with mixed activity are called selective estrogen receptor modulators (SERMS) and are therapeutically useful agents (e.g. EVISTA) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist which sterically hinders the protein sequences required for receptor-coregulatory protein interaction [Pike, et al., Embo 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

The preparation of exemplary ERβ selective ligands, including 2-(3-fluoro-4-hydroxyphenyl)-7-vinyl-1,3-benzoxazol-5-ol (ERB-041), is described in U.S. Pat. No. 6,794,403, incorporated herein by reference in its entirety. Further ERβ selective ligands include compounds set forth in U.S. Pat. No. 6,794,403, U.S. Pat. No. 6,914,074; and U.S. Patent Application Ser. No. 60/637,144, filed Dec. 17, 2004, each of which is incorporated herein by reference in its entirety.

As mentioned above, estrogens affect a panoply of biological processes. In addition, where gender differences have been described (e.g. disease frequencies, responses to challenge, etc), it is possible that the explanation involves the difference in estrogen levels between males and females.

Given the importance of these compounds as pharmaceutical agents, it can be seen that effective formulations for delivery of the compounds is of great import. This invention is directed to these, as well as other, important ends.

### SUMMARY OF THE INVENTION

The present invention provides aqueous pharmaceutical compositions that include an ERβ selective ligand. In some embodiments, the compositions include an ERβ selective ligand, a solubilizer/complexant component, and, optionally, a pH adjusting component.

In some embodiments, the ERβ selective ligand is present in an amount of from about 0.14 μg/mL to about 40 mg/mL; the solubilizer/complexant component is present in an amount of from about 0.00021% (w/v) to about 60% (w/v) of the pharmaceutical composition; and the optional pH adjusting component, when present, is present in a concentration of from about 8.75×10⁻⁷ N to about 1.0 N in the pharmaceutical composition.

In some embodiments, the ERβ selective ligand is present in an amount of from about 0.14 μg/mL to about 10 mg/mL; the solubilizer/complexant component is present in an amount of from about 0.00021% (w/v) to about 15% (w/v) of the pharmaceutical composition; and the optional pH adjusting component, when present, is present in a concentration of from about 8.75×10⁻⁷ N to about 0.0625 N in the pharmaceutical composition.

In some embodiments, the ERβ selective ligand is present in an amount of from about 1 mg/mL to about 40 mg/mL; and the solubilizer/complexant component is present in an amount of from about 1% (w/v) to about 60% (w/v) of the pharmaceutical composition. In some further embodiments, the ERβ selective ligand is present in an amount of from about 5 mg/mL to about 40 mg/mL; and the solubilizer/complexant component is present in an amount of from about 5% (w/v) to about 60% (w/v) of the pharmaceutical composition.

In some embodiments, the ERβ selective ligand is present in an amount of from about 1 mg/mL to about 10 mg/mL; the solubilizer/complexant component is present in an amount of from about 1% (w/v) to about 15% (w/v) of the pharmaceutical composition; and the optional pH adjusting component, when present, is present in a concentration of from about 8.75×10⁻⁷ N to about 0.0625 N in the pharmaceutical composition. In some further embodiments, the ERβ selective ligand is present in an amount of from about 5 mg/mL to about 10 mg/mL; the solubilizer/complexant component is present in an amount of from about 5% (w/v) to about 15% (w/v) of the pharmaceutical composition; and the optional pH adjusting component, when present, is present in a concentration of from about 8.75×10⁻⁷ N to about 0.0625 N in the pharmaceutical composition.

In some embodiments, the ERβ selective ligand is present in an amount of from about 1 mg/mL to about 10 mg/mL; and the solubilizer/complexant component is present in an amount of from about 1% (w/v) to about 15% (w/v) of the pharmaceutical composition. In some further embodiments, the ERβ selective ligand is present in an amount of from about 5 mg/mL to about 10 mg/mL; the solubilizer/complexant component is present in an amount of from about 5% (w/v) to about 15% (w/v) of the pharmaceutical composition.

In some embodiments, the solubilizer/complexant component is selected from cyclodextrins and substituted cyclodextrins, preferably hydroxypropyl beta-cyclodextrin and sulfobutyl ether beta-cyclodextrin, more preferably hydroxypropyl beta-cyclodextrin. In some further embodiments, the pH adjusting component is selected from the group consisting of group I and group II metal hydroxides, for example NaOH and KOH, preferably NaOH.

The invention further provides methods for preparing pharmaceutical compositions of the invention, products of the methods, and methods of using the pharmaceutical compositions of the invention.

### BRIEF DESCRIPTION OF THE FIGURES

FIG. 1 depicts the water solubility of Compound 1 with increasing pH.

FIG. 2 depicts the water solubility of the unionized form of Compound 1 with increasing concentrations of hydroxypropyl-beta-cyclodextrin (HPBCD).

FIG. 3 depicts the water solubility of the ionized form of Compound 1 at pH 9.0 and 10.3 with increasing concentrations of hydroxypropyl-beta-cyclodextrin (HPBCD).

FIG. 4 depicts the effect of serial dilution on a 10 mg/mL (pH 9.2) and 30 mg/mL (pH 10.5) solution of Compound 1 containing 15% hydroxypropyl-beta-cyclodextrin (HPBCD) with Phosphate Buffered Saline as a blood model. The y-axis displays the concentration of Compound 1, while the x-axis displays the pH of the solution. The diamond and circle points represent the data points for the 10 mg/mL and the 30 mg/mL solutions of Compound 1, while the triangle points represent the water solubility of Compound 1.

### DETAILED DESCRIPTION

The present invention provides aqueous pharmaceutical compositions that include an ERβ selective ligand. In some embodiments, the compositions include an ERβ selective ligand, a solubilizer/complexant component, and, optionally, a pH adjusting component. The pharmaceutical compositions of the invention are useful for the administration of ERβ selective ligands, preferably via injection, preferably via intravenous injection.

Generally, the ERβ selective ligand is present in an amount of from about 0.14 μg/mL to about 40 mg/mL of the pharmaceutical composition; or from about 1 mg/mL to about 40 mg/mL of the pharmaceutical composition; from about 5 mg/mL to about 40 mg/mL of the pharmaceutical composition; from about 0.14 μg/mL to about 10 mg/mL of the pharmaceutical composition; from about 1 mg/mL to about 10 mg/mL of the pharmaceutical composition; or from about 5 mg/mL to about 10 mg/mL of the pharmaceutical composition. In some embodiments, the ERβ selective ligand has the Formula I:

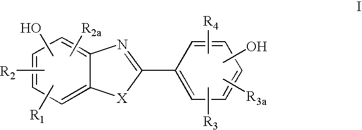

wherein:

R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₅, R₆ are each, independently, hydrogen, alkyl of 1-6 carbon atoms, aryl of 6-10 carbon atoms;

X is O, S, or NR₇; and

R₇ is hydrogen, alkyl of 1-6 carbon atoms, aryl of 6-10 carbon atoms, —COR₅, —CO₂R₅ or —SO₂R₅;

or a pharmaceutically acceptable salt thereof. In some such embodiments, the ERβ selective ligand has the Formula II:

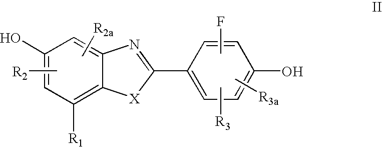

wherein:

R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₅, R₆ are each, independently, hydrogen, alkyl of 1-6 carbon atoms, aryl of 6-10 carbon atoms;

X is O, S, or NR₇; and

R₇ is hydrogen, alkyl of 1-6 carbon atoms, aryl of 6-10 carbon atoms, —COR₅, —CO₂R₅ or —SO₂R₅;

or a pharmaceutically acceptable salt thereof. In some embodiments where the ERβ selective ligand has the Formula II, X is O, and R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In some preferred embodiments, the ERβ selective ligand is 2-(3-fluoro-4-hydroxyphenyl)-7-vinyl-1,3-benzoxazol-5-ol (ERB-041) which has the Formula:

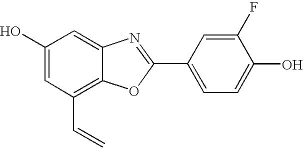

or a pharmaceutically acceptable salt thereof. ERB-041, and compounds of Formulas I and II, can be prepared by the procedures described in U.S. Pat. No. 6,794,403, which is incorporated herein by reference in its entirety.

In some further embodiments, the ERβ selective ligand has the Formula III:

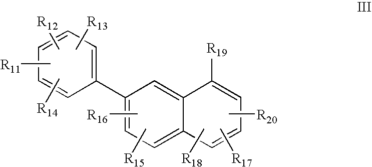

wherein:

R₁₁, R₁₂, R₁₃, and R₁₄ are each, independently, selected from hydrogen, hydroxyl, alkyl of 1-6 carbon atoms, alkoxy of 1-6 carbon atoms, or halogen;

R₁₅, R₁₆, R₁₇, R₁₈, R₁₉, and R₂₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, R₁₉, or R₂₀ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; and

wherein at least one of R₁₁, R₁₂, R₁₃, R₁₄, R₁₇, R₁₈, R₁₉ or R₂₀ is hydroxyl, or a pharmaceutically acceptable salt thereof. In some such embodiments, The ERβ selective ligand has the Formula IV:

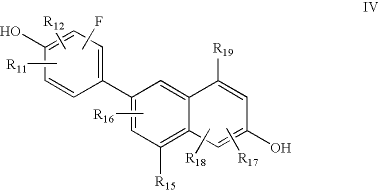

wherein:

R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;

R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; and

wherein at least one of R₁₅ or R₁₉ is not hydrogen, or a pharmaceutically acceptable salt thereof. In some such embodiments, the ERβ selective ligand has the Formula V:

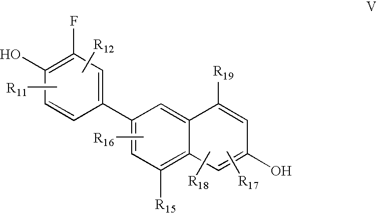

wherein:

R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;

R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; and

wherein at least one of R₁₅ or R₁₉ is not hydrogen, or a pharmaceutically acceptable salt thereof. In some such embodiments, the 5 or 6-membered heterocyclic ring having 1 to 4 heteroatoms selected from O, N or S is furan, thiophene or pyridine, and R₁₅, R₁₆, R₁₇, R₁₈, and R₁₉ are each, independently, hydrogen, halogen, —CN, or alkynyl of 2-7 carbon atoms. In some such embodiments, R₁₆, R₁₇, and R₁₈ are hydrogen. In some embodiments, the ERβ selective ligand is the compound 3-(3-Fluoro-4-hydroxy-phenyl)-7-hydroxy-naphthalene-1-carbonitrile (Compound 1), which has the Formula:

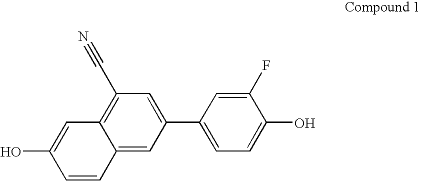

or a pharmaceutically acceptable salt thereof. Compound 1, and compounds of Formulas II, IV and V, can be prepared by the procedures described in U.S. Pat. No. 6,914,074, which is incorporated herein by reference in its entirety.

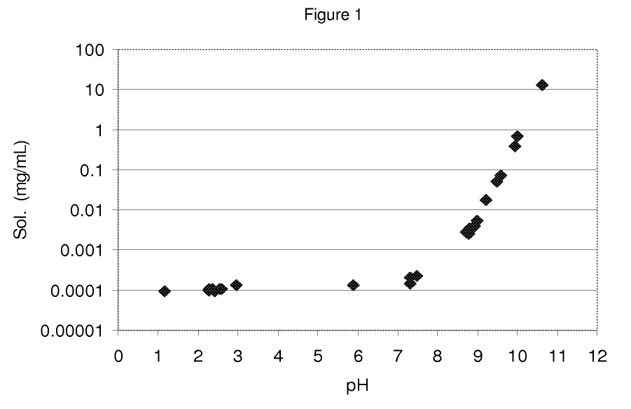

The aqueous pharmaceutical compositions of the invention include a solubilizer/complexant component, to aid in solubilizing the ERβ selective ligand. For example, Compound 1, described above, is insoluble in water, and although an acidic compound, is poorly soluble even at the pH maxima considered safe for IV administration (i.e., about pH 10; see FIG. 1 for the solubility profile of Compound 1). Accordingly, present compositions include a solubilizer/complexant component to aid in solubilization. Generally, the solubilizer/complexant component consists of one or more solubilizing and/or complexing agents known to be useful in the preparation of pharmaceutical formulations. In some embodiments, the solubilizer/complexant component consists of a single solubilizing and/or complexing agent. In some embodiments, the solubilizer/complexant component includes, but is not limited to, cosolvents such as glycerine, ethanol, propylene glycol, sorbitol and polyethylene glycol, and surfactants such as the polyoxyethylene sorbitan fatty acid esters (e.g., polysorbate 80), polyoxyethylene castor oil derivatives (e.g., cremophor EL, cremophor RH40), vitamin E TPGS (d-alpha-tocopheryl polyethylene glycol), solutol (polyethylene glycol esters of hydroxystearate), polyoxyethylene-polyoxypropylene copolymers, polyoxyethylene fatty alcohol ethers, polyethoxylated fatty acid esters, polyoxyethylene-glycerol fatty esters, polyglycolized glycerides, polyethoxylated cholesterols, polyethoxylated sterols, and polyethoxylated vegetable oils. In some embodiments, the solubilizer/complexant component includes, but is not limited to, cosolvents such as glycerine, ethanol, propylene glycol, and polyethylene glycol, and surfactants such as the polyoxyethylene sorbitan fatty acid esters, polyoxyethylene castor oil derivatives, vitamin E TPGS, and solutol. In some embodiments, the solubilizer/complexant component is one or more cyclic oligosaccharides which can be substituted (e.g. with one or more C₁₋₈ alkyl groups, hydroxyl-C₁₋₈-alkyl groups, or sulfo(C₁₋₈-alkyl)ether (MOSO₂—(C₁₋₈-alkyl)-O—) groups (wherein M is a metal salt such as sodium) or unsubstituted. Examples of some preferred solubilizing and/or complexing agents include cyclodextrins (including alpha, beta and gamma cyclodextrins) and substituted cyclodextrins, for example hydroxypropyl beta-cyclodextrin and sulfobutyl ether beta-cyclodextrin, with hydroxypropyl beta-cyclodextrin being preferred. Generally, the solubilizer/complexant component is present in an amount of from about 0.00021% (w/v) to about 60% (w/v) of the pharmaceutical composition; from about 1% (w/v) to about 60% (w/v) of the pharmaceutical composition; from about 5% (w/v) to about 60% (w/v) of the pharmaceutical composition; from about 0.00021% (w/v) to about 15% (w/v) of the pharmaceutical composition; from about 1% (w/v) to about 15% (w/v) of the pharmaceutical composition; or from about 5% (w/v) to about 15% (w/v) of the pharmaceutical composition. In some embodiments, the solubilizer/complexant component does not comprise an anionic or non-ionic surfactant or wetting agent. In some embodiments, the solubilizer/complexant component does not comprise one or more poloxamer 188, benzalkonium chloride, calcium stearate, cetostearyl alcohol, cetomacrogol emulsifying wax, sorbitan esters, or sodium dodecylsulfate; or subembodiments thereof.

As used herein, the term “fatty acid” refers to an aliphatic acid that is saturated or unsaturated. In some embodiments, the fatty acid in a mixture of different fatty acids. In some embodiments, the fatty acid has between about eight to about thirty carbons on average. In some embodiments, the fatty acid has about eight to about twenty-four carbons on average. In some embodiments, the fatty acid has about twelve to about eighteen carbons on average. Suitable fatty acids include, but are not limited to, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, benhenic acid, isobehenic acid, and arachidonic acid, or mixtures thereof. Other suitable fatty alcohols include, but are not limited, the Hystrene® series (available from Humko).

As used herein, the term “fatty alcohol” refers to an aliphatic alcohol that is saturated or unsaturated. In some embodiments, the fatty alcohol in a mixture of different fatty alcohols. In some embodiments, the fatty alcohol has between about eight to about thirty carbons on average. In some embodiments, the fatty alcohol has about eight to about twenty-four carbons on average. In some embodiments, the fatty alcohol has about twelve to about eighteen carbons on average. Suitable fatty alcohols include, but are not limited to, stearyl alcohol, lauryl alcohol, palmityl alcohol, palmitolyl acid, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “fatty ester” refers to an ester compound formed between a fatty acid and an organic compound containing a hydroxyl group.

As used herein, the term “polyethylene glycol” refers to a polymer containing ethylene glycol monomer units of formula —O—CH₂—CH₂—. Suitable polyethylene glycols may have a free hydroxy group at each end of the polymer molecule, or may have one hydroxy group etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer. In some embodiments, the polyethylene glycol is polyethylene glycol-400. Suitable polyethylene glycols include, but are not limited to the Carbowax™ and Carbowax™ Sentry series (available from Dow), the Lipoxol™ series (available from Brenntag), the Lutrol™ series (available from BASF), and the Pluriol™ series (available from BASF).

As used herein, the term “polyethoxylated fatty acid ester” refers to a monoester or diester, or mixture thereof, derived from the ethoxylation of a fatty acid. The polyethoyxylated fatty acid ester can contain free fatty acids and polyethylene glycol as well. Fatty acids useful for forming the polyethoxylated fatty acid esters include, but are not limited to, those described herein. Suitable polyethoxylated fatty acid esters include, but are not limited to, Emulphor™ VT-679 (stearic acid 8.3 mole ethoxylate, available from Stepan Products), the Alkasurf™ CO series (available from Alkaril), macrogol 15 hydroxystearate, Solutol™ HS15 (available from BASF), and the polyoxyethylene stearates listed in R. C. Rowe and P. J. Shesky, Handbook of pharmaceutical excipients, (2006), 5th ed., which is incorporated herein by reference in its entirety.

As used herein, the term “polyethoxylated cholesterol” refers to a compound, or mixture thereof, formed from the ethoxylation of cholesterol.

As used herein, the term “polyglycolized glycerides”, employed alone or in combination with other terms, refers to the products formed from the esterification of polyethylene glycol, glycerol, and fatty acids; the transesterification of glycerides and polyethylene glycol; or the ethoxylation of a glyceride of a fatty acid. As used herein, the term “polyglycolized glycerides” can, alternatively or additionally, refer to mixtures of monoglycerides, diglycerides, and/or triglycerides with monoesters and/or diesters of polyethylene glycol. Polyglycolized glycerides can be derived from the fatty acids, glycerides of fatty acids, and polyethylene glycols described herein. The fatty ester side-chains on the glycerides, monoesters, or diesters can be of any chain length and can be saturated or unsaturated. The polyglycolized glycerides can contain other materials as contaminants or side-products, such as, but not limited to, polyethylene glycol, glycerol, and fatty acids.

As used herein, the term “polyethoxylated vegetable oil” refers to a compound, or mixture of compounds, formed from ethoxylation of vegetable oil, wherein at least one chain of polyethylene glycol is covalently bound to the vegetable oil. In some embodiments, the fatty acids has between about twelve carbons to about eighteen carbons. Suitable polyethoxylated vegetable oils, include but are not limited to, Cremaphor™ EL or RH series (available from BASF), Emulphor™ EL-719 (available from Stepan products), and Emulphor™ EL-620P (available from GAF).

As used herein, the term “polyoxyethylene castor oil derivative”, refers to a compound formed from the ethoxylation of castor oil, wherein at least one chain of polyethylene glycol is covalently bound to the castor oil. The castor oil may be hydrogenated or unhydrogenated. Synonyms for polyethoxylated castor oil include, but are not limited to polyoxyl castor oil, hydrogenated polyoxyl castor oil, mcrogolglyceroli ricinoleas, macrogolglyceroli hydroxystearas, polyoxyl 35 castor oil, and polyoxyl 40 hydrogenated castor oil. Suitable polyethoxylated castor oils include, but are not limited to, the Nikkol™ HCO series (available from Nikko Chemicals Co. Ltd.), Emulphor™ EL-719 (castor oil 40 mole-ethoxylate, available from Stepan Products), the Cremophore™ series (available from BASF), and the Emulgin® RO and HRE series (available from Cognis PharmaLine). Other suitable polyoxyethylene castor oil derivatives include those listed in R. C. Rowe and P. J. Shesky, Handbook of pharmaceutical excipients, (2006), 5th ed., which is incorporated herein by reference in its entirety.

As used herein, the term “polyethoxylated sterol” refers to a compound, or mixture of compounds, derived from the ethoxylation of a sterol molecule. Suitable polyethoyxlated sterols include, but are not limited to, PEG-24 cholesterol ether, Solulan™ C-24 (available from Amerchol); PEG-30 cholestanol, Nikkol™ DHC (available from Nikko); Phytosterol, GENEROL™ series (available from Henkel); PEG-25 phyto sterol, Nikkol™ BPSH-25 (available from Nikko); PEG-5 soya sterol, Nikkol™ BPS-5 (available from Nikko); PEG-10 soya sterol, Nikkol™ BPS-10 (available from Nikko); PEG-20 soya sterol, Nikkol™ BPS-20 (available from Nikko); and PEG-30 soya sterol, Nikkol™ BPS-30 (available from Nikko). As used herein, the term “PEG” refers to polyethylene glycol.

As used herein, the term “polyoxyethylene-glycerol fatty ester” refers to ethoxylated fatty acid ester of glycerine, or mixture thereof. Suitable polyoxyethylene-glycerol fatty esters include, but are not limited to, PEG-20 glyceryl laurate, Tagat™ L (Goldschmidt); PEG-30 glyceryl laurate, Tagat™ L2 (Goldschmidt); PEG-15 glyceryl laurate, Glycerox™ L series (Croda); PEG-40 glyceryl laurate, Glycerox™ L series (Croda); PEG-20 glyceryl stearate, Capmul™ EMG (ABITEC), Aldo MS-20 KFG (Lonza); PEG-20 glyceryl oleate, Tagat™ 0 (Goldschmidt); PEG-30 glyceryl oleate, Tagat™ O₂ (Goldschmidt).

As used herein, the term “polyoxyethylene fatty alcohol ether” refers to an monoether or diether, or mixtures thereof, formed between polyethylene glycol and a fatty alcohol. Fatty alcohols that are useful for deriving polyoxyethylene fatty alcohol ethers include, but are not limited to, those defined herein. In some embodiments, the polyoxyethylene fatty alcohol ether comprises ethoxylated stearyl alcohols, cetyl alcohols, and cetylstearyl alcohols (cetearyl alcohols). Suitable polyoxyethylene fatty alcohol ethers include, but are not limited to, the Brij™ series of surfactants (available from Uniqema), the Cremophor™ A series (available from BASF), the Emulgen™ series (available from Kao Corp.), the Ethosperse™ (available from Lonza), the Ethylan™ series (available from Brenntag), the Plurafac™ series (available from BASF), the Ritoleth™ and Ritox™ series (available from Rita Corp.), the Volpo™ series (available from Croda), and the Texafor™ series. Blends of polyoxyethylene fatty alcohol ethers with other materials are also useful in the invention. Other suitable polyoxyethylene fatty alcohol ethers include those listed in R. C. Rowe and P. J. Shesky, Handbook of pharmaceutical excipients, (2006), 5th ed., which is incorporated herein by reference in its entirety.

As used herein, the term “polyoxyethylene-polyoxypropylene copolymer” refers to a copolymer that has both oxyethylene monomer units and oxypropylene monomer units. Suitable polyoxyethylene-polyoxypropylene copolymers for use in the invention can be of any chain length or molecular weight, and can include branching. The chain ends may have a free hydroxyl groups or may have one or more hydroxyl groups etherified with a lower alkyl or carboxy group. The polyoxyethylene-polyoxypropylene copolymers can also include other monomers which were copolymerized and which form part of the backbone. For example, butylene oxide can be copolymerized with ethylene oxide and propylene oxide to form polyoxyethylene-polyoxypropylene copolymers useful in the present invention. In some embodiments, the polyoxyethylene-polyoxypropylene copolymer is a block copolymer, wherein one block is polyoxyethylene and the other block is polyoxypropylene. Suitable polyoxyethylene-polyoxypropylene copolymers include, but are not limited to, the Pluronic® series of surfactants (available from BASF), and which consist of the group of surfactants designated by the CTFA name of Poloxamer 108, 124, 188, 217, 237, 238, 288, 338, 407, 101, 105, 122, 123, 124, 181, 182, 183, 184, 212, 231, 282, 331, 401, 402, 185, 215, 234, 235, 284, 333, 334, 335, and 403.

Suitable sorbitols include, but are not limited to, Neosorb (available from Roquette), Partech™ SI (available from Merck), Liponic™ 70-NC and 76-NC (available from Lipo Chemical), and Sorbogem™ (available from SPI polyols).

In some embodiments, the pharmaceutical compositions of the invention include a pH adjusting component, that is used to adjust the pH of the composition to a desired value. In some preferred embodiments, the pharmaceutical compositions of the invention are provided at basic pH, for example from about 9 to about 9.3. In some embodiments, the pH adjusting component, when present, is present in a concentration of from about 8.75×10⁻⁷ N to about 1.0 N; or about 8.75×10⁻⁷ N to about 0.0625 N in the pharmaceutical composition. The concentration of pH adjusting component is based on the amount added to the composition and, therefore, includes any portion which later reacts with another component of the composition through acid-base reactions. Accordingly, in some embodiments, such as those wherein the ERP selective ligand is ERB-041 or Compound 1 shown above, the pH adjusting component includes or consists of a base, for example a group I or group II metal hydroxide, for example NaOH and KOH; metal carbonates and bicarbonates, for example, sodium carbonate, potassium carbonate, sodium bicarbonate, or potassium bicarbonate; or an amine base. In some embodiments, the pH adjusting component includes or consists of NaOH or KOH. In some preferred embodiments, the pH adjusting component includes or consists of NaOH. The pH adjusting component can be added as a solid or as a concentrated solution. In some embodiments, the pH component is a base, for example NaOH, added as an aqueous solution.

In some embodiments, the pharmaceutical compositions have greater chemical stability as compared with compositions of the ERβ selective ligands without any solubilizer/complexant component. In some embodiments, the pharmaceutical composition has a potency of the ERβ selective ligand greater than or equal to about 99% at two months at 4° C. In some embodiments, the pharmaceutical composition has a potency of the ERβ selective ligand greater than or equal to about 99.1%, about 99.2%, about 99.3%, about 99.4%, about 99.5%, about 99.6%, about 99.7%, about 99.8%, or about 99.9% at 4° C. As used herein, potency refers to the percent of the initial API concentration.

In some embodiments, the pharmaceutical compositions have less tendency to precipitate as compared with compositions of the ERβ selective ligands without any solubilizer/complexant component. In some embodiments, the pharmaceutical compositions have less tendency to induce phlebitis when administered as compared with compositions of the ERβ selective ligands without any solubilizer/complexant component.

In some embodiments, less than or equal to about 0.1% of the ERβ selective ligand precipitates in two minutes after a 1000-fold dilution of said pharmaceutical composition with phosphate buffered saline. In some embodiments, less than or equal to about 0.01% of the ERβ selective ligand precipitates in two minutes after a 1000-fold dilution of said pharmaceutical composition with phosphate buffered saline. In some embodiments, less than or equal to about 1% or 0.001% of the ERβ selective ligand precipitates in two minutes after a 1000-fold dilution of said pharmaceutical composition with phosphate buffered saline In some embodiments, less than or equal to about 1%, about 0.1%, about 0.01%, or about 0.001% of the ERβ selective ligand precipitates in two minutes after a 100-fold dilution of said pharmaceutical composition with phosphate buffered saline. In some embodiments, no visible precipitate of said ERβ selective ligand is observed in two minutes after a 1000-fold dilution of said pharmaceutical composition with phosphate buffered saline.

The invention further provides methods for preparing pharmaceutical compositions of the invention. In some embodiments, the methods include (i) providing a container (i.e., a vessel suitable for preparing a liquid pharmaceutical composition) including the ERβ selective ligand; (ii) adding the solubilizer/complexant component to the container to form a first mixture; (iii) adding sterile water to the container to form a second mixture; (iv) optionally adding the pH adjustment component to the second mixture to form a third mixture; (v) dissolving the components of the third mixture to form a solution (for example, by stirring, heating, or both stirring and heating); and (vi) filtering the solution.

In some embodiments, the solubilizer/complexant component is present in an amount sufficient to reduce the incidence of phlebitis as compared to administration of a therapeutically effective amount of a pharmaceutical composition of the present invention which does not comprise said solubilizer/complexant component. As used herein, “reduced incidence of phlebitis” means that a statistically significant lower percentage of patients develop phlebitis when administered a therapeutically effective amount of pharmaceutical composition of the present invention as compared to patients administered a therapeutically effective amount of a pharmaceutical composition comprising a ERβ selective ligand (as defined herein) and not comprising a solubilizer/complexant component.

ERβ selective ligands have been disclosed to be useful in the treatment of a variety of diseases and disorders. See U.S. Pat. Nos. 6,794,403 and 6,914,074, supra. Accordingly, the pharmaceutical compositions of the invention find use in the treatment of such diseases and disorders. In some preferred embodiments, the present pharmaceutical compositions are used to treat disorders associated with inflammation or autoimmune diseases, including inflammatory bowel disease (Crohn's disease, ulcerative colitis, indeterminate colitis), arthritis (rheumatoid arthritis, spondyloarthropathies, osteoarthritis), pleurisy, ischemia/reperfusion injury (e.g. stroke, transplant rejection, myocardial infarction, etc.), asthma, giant cell arteritis, prostatitis interstitial cystitis, uveitis, psoriasis, multiple sclerosis, systemic lupus erythematosus and sepsis. The pharmaceutical compositions of the invention are also useful in treating or inhibiting endometriosis.

Accordingly, in some embodiments, the invention provides methods for treating a subject suffering from arthritis or endometriosis, the method comprising administering to said subject a therapeutically effective amount of a pharmaceutical composition of the invention. In some embodiments, the present invention provides a pharmaceutical composition of the invention for use in the methods of treatment described herein.

As used herein the terms “treatment”, “treating”, “treat” and the like are refer to obtaining a desired pharmacologic and/or physiologic effect. The effect may be prophylactic in terms of completely or partially preventing a disease or symptom thereof and/or may be therapeutic in terms of a partial or complete stabilization or cure for a disease, disorder, and/or adverse effect attributable to the disease. “Treatment” as used herein covers any treatment of a disease or disorder in a subject, particularly a human, and includes: (a) preventing the disease or disorder, symptom thereof, from occurring in a subject which may be predisposed to the disease, or disorder, or symptom but has not yet been diagnosed as having it; (b) inhibiting one or more symptoms of such a disease or disorder, i.e., arresting its development; or relieving the symptom of the disease or disorder, i.e., causing regression of the disease, disorder or symptom thereof.

The terms “individual”, “subject”, “host” and “patient” are used interchangeably and refer to any subject for whom diagnosis, treatment, or therapy is desired, particularly humans. Other subjects may include cattle, dogs, cats, guinea pigs, rabbits, rats, mice, horses, and the like. In some preferred embodiments the subject is a human.

As used herein, the terms “administering” or “providing” means directly administering the ERβ selective ligand, preferably via an injection, preferably via intravenous injection.

As used herein, the term “ERβ selective ligand” means a compound wherein the binding affinity (as measured by IC₅₀, where the IC₅₀ of 17β-estradiol is not more than 3 fold different between ERα and ERβ) of the ligand to ERβ is at least about 10 times greater than its binding affinity to ERα in a standard pharmacological test procedure that measures the binding affinities to ERβ and ERα. See U.S. Pat. Nos. 6,794,403 and 6,914,074, incorporated herein by reference in their entirety. In preferred embodiments, the ERβ selective ligand has one of the Formulas I-V described herein, preferably ERB-041 or Compound 1.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. Alkyl groups can contain from 1 to about 20, 1 to about 10, 1 to about 8, 1 to about 6, 1 to about 4, or 1 to about 3 carbon atoms. In some embodiments, alkyl groups can be substituted with up to four substituent groups, as described below. As used herein, the term “lower alkyl” is intended to mean alkyl groups having up to six carbon atoms.

As used herein, “alkenyl” refers to an alkyl group having one or more double carbon-carbon bonds. Example alkenyl groups include ethenyl, propenyl, butenyl, pentenyl, hexenyl, butadienyl, pentadienyl, hexadienyl, and the like. In some embodiments, alkenyl groups can be substituted with up to four substituent groups, as described below.

As used herein, “alkynyl” refers to an alkyl group having one or more triple carbon-carbon bonds. Examples of alkynyl groups include ethynyl, propynyl, butynyl, pentynyl, and the like. In some embodiments, alkynyl groups can be substituted with up to four substituent groups, as described below.

As used herein, “cycloalkyl” refers to non-aromatic carbocyclic groups including cyclized alkyl, alkenyl, and alkynyl groups. Cycloalkyl groups can be monocyclic (e.g., cyclohexyl) or poly-cyclic (e.g. 2, 3, or 4 fused ring, bridged, or spiro monovalent saturated hydrocarbon moiety), wherein the carbon atoms are located inside or outside of the ring system. Any suitable ring position of the cycloalkyl moiety may be covalently linked to the defined chemical structure. Examples of cycloalkyl groups include cyclopropyl, cyclopropylmethyl, cyclobutyl, cyclopentyl, cyclohexyl, cyclohexylmethyl, cyclohexylethyl, cycloheptyl, cyclopentenyl, cyclohexenyl, cyclohexadienyl, cycloheptatrienyl, norbornyl, norpinyl, norcarnyl, adamantyl, spiro[4.5]deanyl, homologs, isomers, and the like. Also included in the definition of cycloalkyl are moieties that have one or more aromatic rings fused (i.e., having a bond in common with) to the cycloalkyl ring, for example, benzo derivatives of cyclopentane (indanyl), cyclohexane (tetrahydronaphthyl), and the like.

As used herein, “hydroxy” or “hydroxyl” refers to OH.

As used herein, “halo” or “halogen” includes fluoro, chloro, bromo, and iodo.

As used herein, “cyano” refers to CN.

As used herein, “alkoxy” refers to an —O-alkyl group. Example alkoxy groups include methoxy, ethoxy, propoxy (e.g., n-propoxy and isopropoxy), t-butoxy, and the like. An alkoxy group can contain from 1 to about 20, 1 to about 10, 1 to about 8, 1 to about 6, 1 to about 4, or 1 to about 3 carbon atoms. In some embodiments, alkoxy groups can be substituted with up to four substituent groups, as described below.

As used herein, the term “perfluoroalkoxy” indicates a group of formula —O-perfluoroalkyl.

As used herein, “haloalkyl” refers to an alkyl group having one or more halogen substituents. Examples of haloalkyl groups include CF₃, C₂F₅, CHF₂, CCl₃, CHCl₂, C₂Cl₅, and the like. An alkyl group in which all of the hydrogen atoms are replaced with halogen atoms can be referred to as “perhaloalkyl.” Examples perhaloalkyl groups include CF₃ and C₂F₅.

As used herein, “haloalkoxy” refers to an —O-haloalkyl group.

As used herein, “aryl” refers to aromatic carbocyclic groups including monocyclic or polycyclic aromatic hydrocarbons such as, for example, phenyl, 1-naphthyl, 2-naphthyl anthracenyl, phenanthrenyl, and the like. In some embodiments, aryl groups have from 6 to about 20 carbon atoms.

As used herein, “heterocyclic ring” is intended to refer to a monocyclic aromatic or non-aromatic ring system having from 5 to 10 ring atoms and containing 1-3 hetero ring atoms selected from O, N and S. In some embodiments, one or more ring nitrogen atoms can bear a substituent as described herein.

As used herein, “arylalkyl” or “aralkyl” refers to a group of formula -alkyl-aryl. Preferably, the alkyl portion of the arylalkyl group is a lower alkyl group, i.e., a C₁₋₆ alkyl group, more preferably a C₁₋₃ alkyl group. Examples of aralkyl groups include benzyl and naphthylmethyl groups.

At various places in the present specification substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, propyl, isopropyl, n-butyl, sec-butyl, isobutyl, etc.

In accordance with the present invention, treatment can also include combination therapy. As used herein “combination therapy” means that the patient in need of treatment is treated or given another drug or treatment modality for the disease in conjunction with the ERβ selective ligand of the present invention. This combination therapy can be sequential therapy where the patient is treated first with one and then the other, or the two or more treatment modalities are given simultaneously. Preferably, the treatment modalities administered in combination with the ERβ selective ligands do not interfere with the therapeutic activity of the ERβ selective ligand.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. It is projected that effective administration of the compositions of the invention may be given to deliver a daily dose of the ERβ selective ligand of from about 5 μg/kg to about 100 mg/kg. The projected daily dosages are expected to vary with route of administration, and the nature of the compound administered.

In some embodiments, the compositions of the invention can be administered to the recipient's bloodstream parenterally (including intravenous, intraperitoneal and subcutaneous injections).

Additional numerous various excipients that are suitable for use in connection with the compositions of the invention are known in the art and described in, for example, Remington's Pharmaceutical Sciences, 17th ed., Mack Publishing Company, Easton, Pa., 1985, which is incorporated herein by reference in its entirety.

### Kits

In some embodiments, a kit comprising a composition of the invention useful for the treatment of the diseases or disorders described herein is provided. The kit comprises a container and a label or package insert on or associated with the container. Suitable containers include, for example, bottles, vials, syringes, etc. The containers can be formed from a variety of materials such as glass or plastic. The container holds or contains a composition of the invention that is effective for treating the disease or disorder of choice and may have a sterile access port (for example the container may be an intravenous solution bag or a vial having a stopper pierceable by a hypodermic injection needle). The article of manufacture can further include a second container having a pharmaceutically acceptable diluent buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. All publications, patent applications, patents, and other references mentioned herein are incorporated by reference in their entirety.

### EXAMPLE 1

### Preparation of 100 mL of an Aqueous Formulation Containing 10 mg/mL of Compound 1 in 15% Hydroxypropyl-beta-cyclodextrin (HPBCD)/0.06N NaOH pH 9.1

1. 1.0 g of Compound 1 was weighed into a tared container.

2. 15.00 g of HPBCD was weighed out and transfer to the container.

3. 82.35 g Sterile Water for Injection was added to the container.

4. 6.25 g (6 mL) of 1 N NaOH was added to the container.

5. The contents of the container were mixed by continuous stirring to dissolve the solids. Up to 30 minutes may be required to completely dissolve the Compound 1.

6. When dissolution was complete, the pH was confirmed to be 9.0-9.3.

7. The solution was then filtered through a Millipore Millex-GV 0.22 u PVDF filter.

8. The final pH was then reconfirmed to be 9.1.

### The composition of the Formulation is shown below in Table 1.

TABLE 1

| | Percent | |
|---|---|---|
| | Composition | |
| Ingredient | (w/v) | Quantity in 100 mL |
| Compound 1 | 1.00 | 1.00 g |
| Hydroxypropyl-beta-cyclodextrin | 15.00 | 15.00 g |
| NaOH 1N | 6.25 | 6.25 g (=6 mL) |
| Sterile water for Injection | qs | 82.35 g |
| Total | | 104.6 g = 100 mL |

The density of the final solution was 1.046 g/mL

Preferably, the above formulation is used at a maximum dose volume of 1 mL/kg.

To obtain lower concentrations for the administration of lower doses or larger dose volumes, the 10 mg/mL formulation above can be diluted, preferably with D5W (dextrose 5% in water). It is preferred that the diluted formulation be used within 24 hours. If the formulation is to be used for more than 24 hours, it should be stored at 4° C. Generally, the formulation can be used for one day when stored at room temperature, and up to seven 7 days when stored at 4° C.

In some embodiments, the formulations can additional contain one or more preservatives, to increase shelf life. Exemplary preservatives are described in Remington's Pharmaceutical Sciences, 17th ed., Mack Publishing Company, Easton, Pa., 1985, which is incorporated herein by reference in its entirety.

### EXAMPLE 2

### Solubility of the Pharmaceutical Compositions of Compound 1

Compound 1 is relatively insoluble at lower pH (see FIG. 1 for the solubility profile). While the Compound 1 can be made soluble at a high pH, IV administration poses a high risk of precipitation of the drug upon dilution in the buffered environment of blood plasma which has a pH of approximately 7.35 to 7.45. This can lead to phlebitis, i.e., inflammation of a vein (see Yalkowsky et al., J. Pharm Sci, 87 (7) 1998, p. 787-796). Therefore, experiments were undertaken to assess whether the compositions of the present invention showed any improvement in the solubility of Compound 1, which would reduce the tendency for phlebitis.

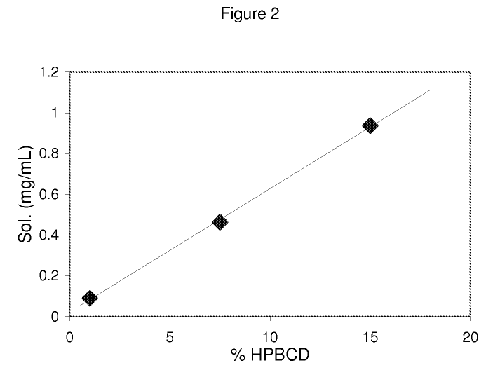

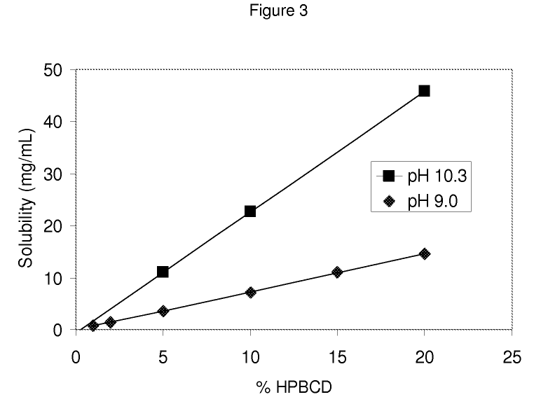

When formulated with 15% HPBCD at pH 9 and 10.3, Compound 1 showed 10-fold and 30-fold increases in solubility, respectively, when compared with the free acid form of Compound 1 at a pH of 6.3-7.6, formulated with 15% HPBCD. For example, FIG. 2 shows the solubility of the free acid form of Compound 1 formulated with varying amounts of HPBCD, while FIG. 3 shows the solubility of the ionized form of Compound 1 formulated with varying amounts of HPBCD, at both pH 9 and pH 10.3.

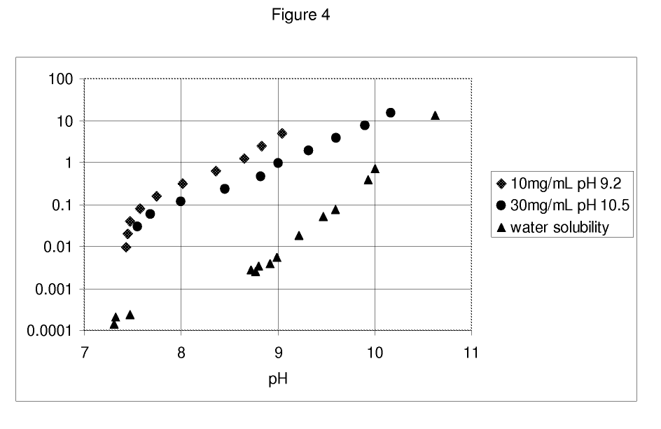

A serial dilution study was also undertaken to assess the risk of Compound 1 precipitating upon injection. Phosphate buffered saline (PBS) was used as the diluent and blood model. Accordingly, 10 mg/mL (at pH 9.2) and 30 mg/mL (at pH 10.5) compositions of Compound 1 were prepared by the method described in Example 1, both having 15% HPBCD. The solutions were then serially diluted with PBS. The PBS solutions were observed for two minutes after dilution for any precipitate (the two minute interval is sufficient for complete dilution in the blood stream). The presence of any precipitate was noted for each diluted solution. Tables 2 and 3 show the results of the study for the 10 mg/mL and 30 mg/mL compositions, respectively, while FIG. 4 shows the effect of dilution on the concentration of Compound 1. The 10 mg/mL solution showed a lower tendency to precipitate upon dilution, likely due to the higher HPBCD to Compound 1 ratio. FIG. 4 shows that the concentration of Compound 1 are maintained above the water solubility for sufficient time for complete dilution in the blood stream when administered.

TABLE 2

| pH | [Compound 1] (mg/mL) | Precipitate at 2 min. |
|---|---|---|
| 7.43 | 0.009766 | none |
| 7.45 | 0.019531 | none |
| 7.47 | 0.039063 | none |
| 7.57 | 0.078125 | none |
| 7.75 | 0.15625 | none |
| 8.01 | 0.3125 | none |
| 8.36 | 0.625 | none |
| 8.65 | 1.25 | none |
| 8.83 | 2.5 | none |
| 9.04 | 5 | none |

TABLE 3

| pH | [Compound 1] (mg/mL) | Precipitate at 2 min. |
|---|---|---|
| 7.55 | 0.029297 | yes |
| 7.68 | 0.058594 | yes |
| 8 | 0.117188 | yes |
| 8.45 | 0.234375 | yes |
| 8.82 | 0.46875 | none |
| 9 | 0.9375 | none |
| 9.32 | 1.875 | none |
| 9.6 | 3.75 | none |
| 9.9 | 7.5 | none |
| 10.17 | 15 | none |

### EXAMPLE 3

### Chemical Stability of a Pharmaceutical Composition of Compound 1

A pharmaceutical composition of Compound 1 formulated with 15% HPBCD at pH 9.2 was stored at 4° C., 25° C., and 40° C. for six months. Each stored composition was examined at one, two, and six months for stability. The potency of each composition was determined, and the concentration of impurities from degradation was measured by HPLC, as shown in Table 4. The degradation at six months (25° C.) is comparable to what is seen in only three days for a comparable formulation without the HPBCD (5 mg/mL, 50 mM glycine buffer, pH 11), which showed 0.34% of the molecular weight 556 impurity at three days.

TABLE 4

| Storage | Temp. | | Impurity | Im- | Impurity | Impurity |
|---|---|---|---|---|---|---|
| Time | (° C.) | Potency | MW 279 | purity | MW 574 | MW 556 |
| 1 mo. | 40 | 100.4 | 0.07 | 0.06 | 0.03 | 0.33 |
| 1 mo. | 25 | 100.8 | | | | |
| 1 mo. | 4 | ND | ND | ND | ND | ND |
| 3 mo. | 40 | 98.1 | 0.16 | 0.12 | 0.06 | 0.62 |
| 3 mo. | 25 | 98.9 | 0.03 | 0.02 | | 0.11 |
| 3 mo. | 4 | 99.9 | | | | |
| 6 mo. | 40 | 93.3 | 0.4 | 0.3 | 0.11 | 1.57 |
| 6 mo. | 25 | 97.7 | 0.05 | 0.04 | 0.03 | 0.36 |
| 6 mo. | 4 | 98.0 | | | | 0.06 |

Various modifications of the invention, in addition to those described herein, will be apparent to those skilled in the art from the foregoing description. Such modifications are also intended to fall within the scope of the appended claims. Each of the publications and, references, including books and patents, cited in the present application is incorporated herein by reference in its entirety.

What is claimed is:

1. An aqueous pharmaceutical composition comprising:
a) an ERβ selective ligand in an amount of from about 0.14 μg/mL to about 40 mg/mL;
b) a solubilizer/complexant component comprising from about 0.00021% (w/v) to about 60% (w/v) of the pharmaceutical composition; and
c) an optional pH adjusting component in a concentration of from about 8.75×10⁻⁷ N to about 1.0 N;
wherein the ERβ selective ligand has the Formula I: 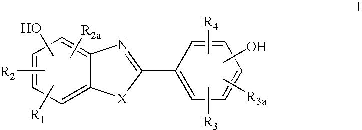
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R_{3ax, and R}₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅;
or a pharmaceutically acceptable salt thereof;
or the Formula III: 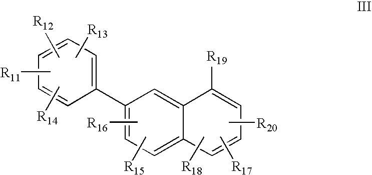
wherein:
R₁₁, R₁₂, R₁₃, and R₁₄ are each, independently, selected from hydrogen, hydroxyl, alkyl of 1-6 carbon atoms, alkoxy of 1-6 carbon atoms, or halogen;
R₁₅, R₁₆, R₁₇, R₁₈, R₁₉, and R₂₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, R₁₉, or R₂₀ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; and
wherein at least one of R₁₁, R₁₂, R₁₃, R₁₄, R₁₇, R₁₈, R₁₉ or R₂₀ is hydroxyl, or a pharmaceutically acceptable salt thereof.

2. The pharmaceutical composition of claim 1, wherein:
said ERβ selective ligand is present in an amount of from about 0.14 μg/mL to about 10 mg/mL;
said solubilizer/complexant component comprises from about 0.00021% (w/v) to about 15% (w/v) of the pharmaceutical composition; and
said optional pH adjusting component in an amount of about 8.75×10⁻⁷ N to about 0.0625 N.

3. The pharmaceutical composition of claim 1, wherein:
said ERβ selective ligand is present in an amount of from about 1 mg/mL to about 40 mg/mL; and
said solubilizer/complexant component is present in an amount of from about 1% (w/v) to about 60% (w/v) of the pharmaceutical composition.

4. The pharmaceutical composition of claim 1, wherein:
said ERβ selective ligand is present in an amount of from about 5 mg/mL to about 40 mg/mL; and
said solubilizer/complexant component is present in an amount of from about 5% (w/v) to about 60% (w/v) of the pharmaceutical composition.

5. The pharmaceutical composition of claim 1, wherein:
said ERβ selective ligand is present in an amount of from about 1 mg/mL to about 10 mg/mL;
said solubilizer/complexant component is present in an amount of from about 1% (w/v) to about 15% (w/v) of the pharmaceutical composition; and
said optional pH adjusting component in an amount of about 8.75×10⁻⁷ N to about 0.0625 N.

6. The pharmaceutical composition of claim 1, wherein:
said ERβ selective ligand is present in an amount of from about 5 mg/mL to about 10 mg/mL;
said solubilizer/complexant component is present in an amount of from about 5% (w/v) to about 15% (w/v) of the pharmaceutical composition; and
said optional pH adjusting component in an amount of about 8.75×10⁻⁷ N to about 0.0625 N.

7. The pharmaceutical composition of claim 1, wherein the ERβ selective ligand has the Formula II: 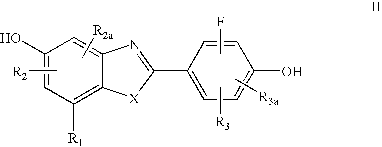
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅;
or a pharmaceutically acceptable salt thereof.

8. The pharmaceutical composition of claim 7, wherein the ERβ selective ligand has the Formula II, wherein X is O, and R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆.

9. The pharmaceutical composition of claim 2, wherein the ERβ selective ligand has the Formula II: 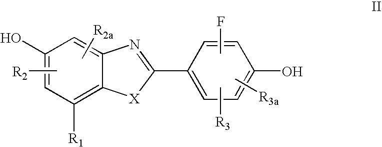
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅;
or a pharmaceutically acceptable salt thereof.

10. The pharmaceutical composition of claim 1, wherein the ERβ selective ligand has the Formula: 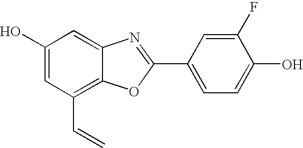
or a pharmaceutically acceptable salt thereof.

11. The pharmaceutical composition of claim 10, wherein the solubilizer/complexant component is selected from the group consisting of hydroxypropyl beta-cyclodextrin and sulfobutyl ether beta-cyclodextrin.

12. The pharmaceutical composition of claim 10, wherein the solubilizer/complexant component comprises hydroxypropyl beta-cyclodextrin.

13. The pharmaceutical composition of claim 10, wherein the pH adjusting component is selected from the group consisting of group I and group II metal hydroxides.

14. The pharmaceutical composition of claim 10, wherein the pH adjusting component is selected from the group consisting of NaOH and KOH.

15. The pharmaceutical composition of claim 10, wherein:
the solubilizer/complexant component is selected from the group consisting of hydroxypropyl beta-cyclodextrin and sulfobutyl ether beta-cyclodextrin; and
the pH adjusting component is selected from the group consisting of group I and group II metal hydroxides.

16. The pharmaceutical composition of claim 2, wherein the ERβ selective ligand has the Formula: 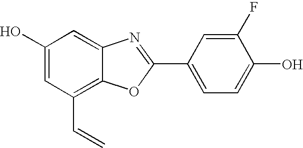
or a pharmaceutically acceptable salt thereof.

17. The pharmaceutical composition of claim 1, wherein the ERβ selective ligand has the Formula IV: 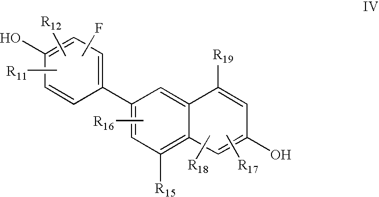
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; and
wherein at least one of R₁₅ or R₁₉ is not hydrogen, or a pharmaceutically acceptable salt thereof.

18. The pharmaceutical composition of claim 17, wherein the ERβ selective ligand has the Formula V: 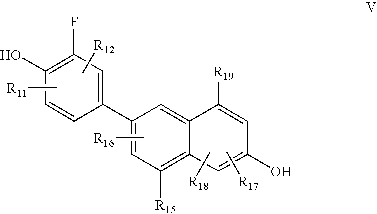
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; and
wherein at least one of R₁₅ or R₁₉ is not hydrogen, or a pharmaceutically acceptable salt thereof.

19. The pharmaceutical composition of claim 18, wherein the 5 or 6-membered heterocyclic ring having 1 to 4 heteroatoms selected from O, N or S is furan, thiophene or pyridine, and R₁₅, R₁₆, R₁₇, R₁₈, and R₁₉ are each, independently, hydrogen, halogen, —CN, or alkynyl of 2-7 carbon atoms.

20. The pharmaceutical composition of claim 18, wherein R₁₆, R₁₇, and R₁₈ are hydrogen.

21. The pharmaceutical composition of claim 2, wherein the ERβ selective ligand has the Formula IV: 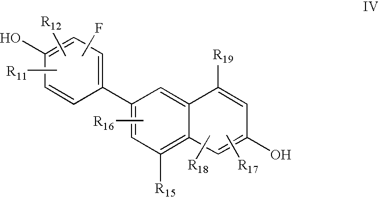
wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; and
wherein at least one of R₁₅ or R₁₉ is not hydrogen, or a pharmaceutically acceptable salt thereof.

22. The pharmaceutical composition of claim 1, wherein the ERβ selective ligand is a compound having the Formula: 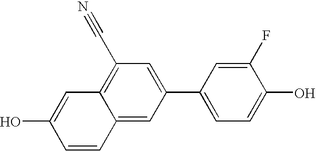
or a pharmaceutically acceptable salt thereof.

23. The pharmaceutical composition of claim 22, wherein the solubilizer/complexant component is selected from the group consisting of hydroxypropyl beta-cyclodextrin and sulfobutyl ether beta-cyclodextrin.

24. The pharmaceutical composition of claim 22, wherein the solubilizer/complexant component comprises hydroxypropyl beta-cyclodextrin.

25. The pharmaceutical composition of claim 22, wherein the pH adjusting component is selected from the group consisting of group I and group II metal hydroxides.

26. The pharmaceutical composition of claim 22, wherein the pH adjusting component is selected from the group consisting of NaOH and KOH.

27. The pharmaceutical composition of claim 22, wherein:
the solubilizer/complexant component is selected from the group consisting of hydroxypropyl beta-cyclodextrin and sulfobutyl ether beta-cyclodextrin; and
the pH adjusting component is selected from the group consisting of group I and group II metal hydroxides.

28. The pharmaceutical composition of claim 27, wherein the pH adjusting component is selected from the group consisting of NaOH and KOH.

29. The pharmaceutical composition of claim 27, wherein the solubilizer/complexant component is hydroxypropyl beta-cyclodextrin; and the pH adjusting component comprises NaOH.

30. The pharmaceutical composition of claim 2, wherein the ERβ selective ligand is a compound having the Formula: 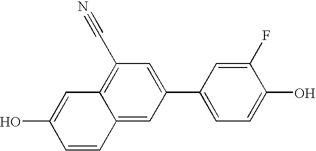
or a pharmaceutically acceptable salt thereof.

31. The pharmaceutical composition of claim 1, wherein the solubilizer/complexant component is selected from cyclodextrins and substituted cyclodextrins.

32. The pharmaceutical composition of claim 1, wherein the solubilizer/complexant component is selected from the group consisting of hydroxypropyl beta-cyclodextrin and sulfobutyl ether beta-cyclodextrin.

33. The pharmaceutical composition of claim 1, wherein the solubilizer/complexant component comprises hydroxypropyl beta-cyclodextrin.

34. The pharmaceutical composition of claim 1, wherein the pH adjusting component is selected from the group consisting of group I and group II metal hydroxides.

35. The pharmaceutical composition of claim 1, wherein the pH adjusting component is selected from the group consisting of NaOH and KOH.

36. The pharmaceutical composition of claim 1, wherein:
the solubilizer/complexant component is selected from the group consisting of hydroxypropyl beta-cyclodextrin and sulfobutyl ether beta-cyclodextrin; and
the pH adjusting component is selected from the group consisting of group I and group II metal hydroxides.

37. The pharmaceutical composition of claim 36, wherein the pH adjusting component is selected from the group consisting of NaOH and KOH.

38. The pharmaceutical composition of claim 36, wherein the solubilizer/complexant component is hydroxypropyl beta-cyclodextrin; and the pH adjusting component comprises NaOH.

39. The pharmaceutical composition of claim 36, wherein the pH adjusting component is selected from the group consisting of NaOH and KOH.

40. The pharmaceutical composition of claim 36, wherein the solubilizer/complexant component is hydroxypropyl beta-cyclodextrin; and the pH adjusting component comprises NaOH.

41. The pharmaceutical composition of claim 1, wherein:
the solubilizer/complexant component is selected from the group consisting of hydroxypropyl beta-cyclodextrin and sulfobutyl ether beta-cyclodextrin; and
the ERβ selective ligand has the Formula: 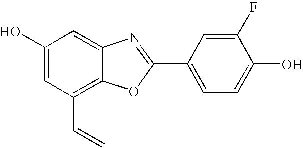
or a pharmaceutically acceptable salt thereof;
or the Formula: 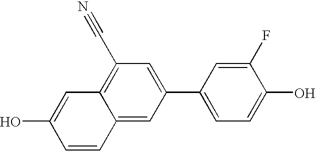
or a pharmaceutically acceptable salt thereof.

42. The pharmaceutical composition of claim 41, wherein the solubilizer/complexant component comprises hydroxypropyl beta-cyclodextrin.

43. The pharmaceutical composition of claim 42, wherein the pH adjusting component is selected from the group consisting of group I and group II metal hydroxides.

44. The pharmaceutical composition of claim 42, wherein the pH adjusting component is selected from the group consisting of NaOH and KOH.

45. The pharmaceutical composition of claim 42, wherein the pH adjusting component comprises NaOH.

46. The pharmaceutical composition of claim 42, wherein:
said ERβ selective ligand is present in an amount of from about 5 mg/mL to about 10 mg/mL; and
said solubilizer/complexant component is present in an amount of from about 5% (w/v) to about 15% (w/v) of the pharmaceutical composition.

47. The pharmaceutical composition of claim 42, wherein:
said ERβ selective ligand is present in an amount of about 10 mg/mL; and
said solubilizer/complexant component is present in an amount of about 15% (w/v) of the pharmaceutical composition.

48. The pharmaceutical composition of claim 1 having a potency of the ERβ selective ligand in said pharmaceutical composition greater than or equal to about 99% at two months at 4° C.

49. The pharmaceutical composition of claim 1, wherein less than or equal to about 0.01% of the ERβ selective ligand precipitates in two minutes after a 1000-fold dilution of said pharmaceutical composition with phosphate buffered saline.

50. The pharmaceutical composition of claim 1, wherein less than or equal to about 0.1% of the ERβ selective ligand precipitates in two minutes after a 1000-fold dilution of said pharmaceutical composition with phosphate buffered saline.

51. A method for preparing a pharmaceutical composition of claim 1, the method comprising:
(i) providing a container comprising said ERβ selective ligand;
(ii) adding said solubilizer/complexant component to said container to form a first mixture;
(iii) adding sterile water to said container to form a second mixture;
(iv) add said pH adjustment component to said second mixture to form a third mixture;
(v) dissolving the components of said third mixture to form a solution; and
(vi) filtering said solution.

52. A product of the process of claim 51.

53. A method for treating a subject suffering from arthritis or endometriosis, the method comprising administering to said subject a therapeutically effective amount of a pharmaceutical composition of claim 1.

54. The method of claim 53, wherein said solubilizer/complexant component is present in an amount sufficient to reduce the incidence of phlebitis as compared to administration of a therapeutically effective amount of a pharmaceutical composition of claim 1 which does not comprise said solubilizer/complexant component.

55. A kit comprising a composition of claim 1, and container therefor.
